(19) United States
(12) Patent Application Publication
(10) Pub. No.: US 2002/0095213 A1
(43) Pub. Date: Jul. 18, 2002

(54) PROSTHETIC DEVICES FORMED FROM MATERIALS HAVING BONE-BONDING PROPERTIES AND USES THEREFOR

(76) Inventors: DIRKJAN BAKKER, ALPHEN AAN DEN (NL); JOHANNES J. GROTE, ZOETERWOEDE (NL); CLEMENS A. VAN BLITTERSWIJK, HEKENDORP (NL)

Correspondence Address: RAYMOND J. LILLIE, CARELLA, BYRNE, BAIN, GILFILLAN,, CECCHI & STEWART, 6 BECKER FARM ROAD, ROSELAND, NJ, 07068

(21) Appl. No.: 08/089,854

(22) Filed: Jul. 12, 1993

Related U.S. Application Data
(63) Continuation-in-part of application No. 07/907,674, filed on Jul. 2, 1992, now abandoned.
Application No. 07/907,674 is a continuation-in-part of application No. 07/479,197, filed on Feb. 13, 1990, now abandoned.
Application No. 07/479,197 is a continuation-in-part of application No. 07/240,810, filed on Sep. 2, 1988, now abandoned.

(30) Foreign Application Priority Data
Sep. 2, 1988 (NL) 882178

(51) Int. Cl. A61F 2/08; A61F 2/28; C08G 63/66

(52) U.S. Cl. 623/13.11; 623/23.58; 433/201.1; 528/301; 523/115

(57) ABSTRACT

A prosthetic device formed from a polymer which, when contacted with a calcium salt, calcium is deposited on or in the polymer. The polymer includes a soft component and a hard component. The device has bone-bonding properties. The soft component provides for the deposition of calcium on or in the soft component and preferably is a polyalkylene glycol, and the hard component preferably is a polyester. A preferred material is a polyethylene glycol/polybutylene terephthalate copolymer.

This application is a continuation-in-part of application Ser. No. 907,674, filed Jul. 2, 1992, which is a continuation-in-part of application Ser. No. 479,197, filed Feb. 13, 1990, now abandoned, which is a continuation-in-part of application Ser. No. 240,810, filed Sep. 2, 1988, now abandoned.

This invention relates to prosthetic devices having bone-bonding properties. More particularly, this invention relates to prosthetic devices comprised of a polymer, which, when contacted with calcium (such as in the form of a calcium salt in aqueous solution), calcium is deposited on or in the polymer.

It has been known to form prosthetic devices from non-elastomeric materials such as, for example, bioglasses, glass ceramics, and calcium phosphate (eg., “hydroxyapatite”) ceramics. The ceramic “hydroxyapatite” is bioactive as concerns bonding to bone (C. A. van Blitterswijk et al., “Bioreactions at the tissue/hydroxyapatite interface”, Biomaterials, Vol. 6, pages 243-251 (1985). The so-called “lamina limitans”-like interface (LL-interface) at the interface between hydroxyapatite and bone which inorganic part mainly consists of hydroxyapatite, is characteristic for the chemical bond between both materials. In particular, said chemical bond is thought to be based on a bilateral crystal growth. The sintered hydroxyapatite, however, belongs to the ceramics which are non-elastic materials.

U.S. Pat. No. 3,908,201, issued to Jones, et al., discloses a prosthetic device which binds to collagenous body tissue. The prosthetic device is formed from a plastic material which is a copolymer of polyethylene glycol and a component which stabilizes the material in water, such as an ester, a urethane, or an amide. Preferred materials are copolymers of polyethylene glycol and polyethylene terephthalate and copolymers of polyethylene glycol and bis-(β-hydroxyethyl) terephthalate or isophthalate. The patent does not disclose or suggest that such plastic materials bind to hard tissue, such as bone.

In accordance with an aspect of the present invention, there is provided a prosthetic device comprised of a polymer which, when contacted with calcium (in particular a calcium salt in aqueous solution such as, but not limited to, calcium phosphate), calcium is deposited on or in the polymer. The deposition of calcium can result from or be accomplished by absorption, adsorption, precipitation, chelation, etc. The polymer is biocompatible, and preferably is synthetic. The ability of the polymer to provide for the deposition of calcium is believed to result in the bonding of the polymer to bone.

Although the scope of the present invention is not to be limited to any theoretical reasoning, it is believed that calcium ions and other ions in solution (such as phosphate ions) whether contained in in vitro fluids or in body fluids in vivo, diffuse into the polymer and are deposited on or in the polymer as a calcium salt (such as calcium phosphates; eg., monotite (CaHPO₄), brushite [CaHPO₄.2H₂O], tricalcium phosphate, or tetracalcium phosphate, or hydroxyapatite, for example).

The highest ion concentrations occur, in general, at the surface region of the polymer. The calcium phosphates recrystallize and organize in the polymer, and an electron-dense interface layer between the bone and the newly-formed calcium salts develops at the surface of the polymer as deposition of cells and cell-derived materials (eg., proteins) occurs. Star-like shaped needles of the deposited calcium salts are also formed; such needles become locked into the surface of the polymer, thereby forming a bond between the deposited calcium salts and the natural bone surface. Thus, the polymer establishes a tight chemical bond between the polymer and the bone at the molecular level and the physical level.

More particularly, the device is comprised of a copolymer which includes two components. The first component is a component which, when contacted with calcium (in particular in the form of a calcium salt), calcium is deposited on or in the first component (such as, for example, in the form of a calcium salt such as calcium phosphate). The first component also preferably is capable of absorbing water. The second component is non-water absorbing (i.e., hydrophobic) and provides water resistance.

The first component is a so-called “soft” component and the second component is a so-called “hard” component. The soft component, which provides the material with its biological properties, (eg., bone bonding), may be present in an amount of from about 20 wt. % to about 98 wt. % of the polymer, preferably from about 40 wt. % to about 80 wt. %. In general, the polymer becomes more elastomeric as the amount of the soft component increases. Also, as the amount of soft component increases, the rate of calcification (i.e., deposition of calcium on or in the polymer), increases as well. As the amount of soft component decreases, the rigidity of the material increases, and the rate and amount of calcification and bone bonding decreases. In a preferred embodiment, the soft component is in the form of a hydrogel.

The soft component may include a component which may be selected from the group consisting of polyethers (both substituted and unsubstituted); polyamines; polyvinyl acetate; polyvinyl alcohol; polyvinyl pyrrolidone; polyacrylic acid; poly (hydroxyethyl methacrylate); thioethers; and polypentapeptides of elastin.

The polypentapeptides of elastin include a repeat pentapeptide sequence, and may be selected from the group consisting of:

(Val Pro Gly Val Gly)ₙ Val

(Gly Val aly Val Pro)ₙ; and

(Gly Val Gly Val Pro)ₙ Val,

wherein n is at least 2, preferably from about 10 to about 240. The pentapeptide units, in a preferred embodiment, are cross-linked with gamma radiation. Such polypentapeptides are further described in Wood, et al., J. Biol. Mater. Res., Vol. 20, pgs. 315-335 (1986).

In one embodiment, the soft component includes a polyether, preferably a polyalkylene glycol. The polyalkylene glycol may be selected from the group consisting of polyethylene glycol, polypropylene glycol, and polybutylene glycol. In one embodiment, the polyalkylene glycol is polyethylene glycol.

The hard component may be present in the polymer in an amount of from about 2 wt. % to about 80 wt. %, preferably from about 20 wt. % to about 60 wt. %. The hard component stabilizes the soft component in water, as well as provide the physical characteristics of the polymer, and provides mechanical strength to the polymer. Although the scope of the present invention is not to be limited thereby, the hard component may form crystallites which prevent the soft component from dissolving into the body. Thus, the soft component remains stable and thus permits deposition of calcium salts upon the soft component.

The hard component may be selected from the group consisting of urethanes, amides, and esters. The ester may have the following structural formula:

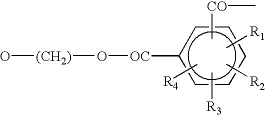

, wherein n is from 2 to 8, and each of R₁, R₂, R₃, and R₄ is hydrogen, chlorine, nitro-, or alkoxy, and each of R₁, R₂, R₃, and R₄ is the same or different. Alternatively, the ester is derived from a binuclear aromatic diacid having the following structural formula:

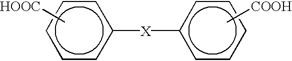

, wherein X is —O—, —SO₂—, or —CH₂—.

Preferably, the hard component is an ester having the following structural formula:

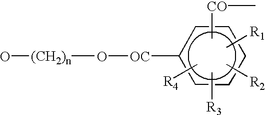

, wherein n is from 2 to 8, and each of R₁, R₂, R₃, and R₄ is hydrogen, chlorine, nitro-, or alkoxy, and each of R₁, R₂, R₃, and R ₄ is the same or different. More preferably, each of R₁, R₂, R₃, and R₄ is hydrogen.

In another embodiment, the ester is polylactic acid.

In yet another embodiment, the ester is polyglycolic acid.

In a preferred embodiment, the polymer is a segmented thermoplastic polymer comprising a plurality of recurring units of the first component and units of the second component. The first component comprises from about 20 wt. % to about 98 wt. %, based upon the weight of the polymer, of units of the formula:

—OLO—CO—R—CO—, wherein L is selected from the group consisting of a divalent radical remaining after removal of terminal hydroxyl groups from a poly (oxyalkylene) glycol; and a polymer including a first moiety and a second moiety, said first moiety being a polyalykylene glycol and said second moiety being selected from the group consisting of glycine anhydride, alloxan, uracil, 5,6-dihydrouracil, glycolic acid, lactic acid, and lactones, such as, for example, dicarboxylic acid lactones. The second component is present in an amount of from about 2 wt. % to about 80 wt. %, based on the weight of the polymer, and is comprised of units of the formula:

—OEO—CO—R—CO—. E is an organic radical selected from the group consisting of a substituted or unsubstituted alkylene radical having from 2 to 8 carbon atoms, and a substituted or unsubstituted ether moiety.

R is a substituted or unsubstituted divalent radical remaining after removal of carboxyl groups from a dicarboxylic acid.

In one embodiment, L is a divalent radical remaining after removal of terminal hydroxyl groups from a poly (oxyalkylene) glycol. The poly (oxyalkylene) glycol, in one embodiment, may be selected from the group consisting of poly (oxyethylene) glycol, poly (oxypropylene) glycol, and poly (oxybutylene) glycol. Preferably, the poly (oxyalkylene) glycol is poly (oxyethylene) glycol. The poly (oxyethylene) glycol may have a molecular weight of from about 300 to about 12,000, preferably from about 500 to about 6,000, more preferably from about 500 to about 4,000.

In another embodiment, L is a polymer including a first moiety, which is a polyalkylene glycol and a second moiety selected from the group consisting of glycine anhydride, alloxan, uracil, 5,6-dihydrouracil, glycolic acid, lactic acid, and lactones, such as, for example, dicarboxylic acid lactones.

In one embodiment, the polyalkylene glycol moiety is selected from the group consisting of polyethylene glycol, polypropylene glycol, and polybutylene glycol. Preferably, the polyalkylene glycol is polyethylene glycol.

The polyethylene glycol may have a molecular weight of from about 300 to about 12,000, preferably from about 500 to about 6,000, more preferably from about 500 to about 4,000.

In another embodiment, the second moiety is a lactone, and preferably the lactone is D,L-isocitric acid lactone. Thus, in a preferred embodiment, the first moiety is polyethylene glycol and the second moiety is D,L-isocitric acid lactone.

In one embodiment, E is an alkylene radical having from 2 to 8 carbon atoms.

Preferably, E is an alkylene radical having from 2 to 4 carbon atoms, and more preferably the second component is a terephthalate selected from the group consisting of polyethylene terephthalate, polypropylene terephthalate, and polybutylene terephthalate. In one embodiment, the second component is polybutylene terephthalate. The terephthalate may be substituted or unsubstituted.

In a most preferred embodiment, the polymer is a polyethylene glycol/polybutylene terephthalate copolymer.

In one embodiment, the polyethylene glycol/polybutylene terephthalate copolymer may be synthesized from a mixture of dimethylterephthalate, butanediol (in excess), polyethylene glycol, optionally an antioxidant, and a catalyst. The mixture is placed in a reaction vessel and heated to about 180° C., and methanol is distilled as transesterification occurs. During the transesterification, the ester bond with methyl is replaced with an ester bond with butyl. In this step the polyethylene glycol does not react. After transesterification, the temperature is slowly raised to about 245° C. and a vacuum (finally less than 0.1 mbar) is achieved. The excess butanediol is distilled and a prepolymer of butanediol terephthalate condenses with the polyethylene glycol to form a polyethylene glycol/polybutylene terephthalate copolymer. A terephthalate moiety connects the polyethylene glycol units to the polybutylene terephthalate units of the copolymer, and thus such copolymer is also sometimes hereinafter referred to as a polyethylene glycol terephthalate/polybutylene terephthalate copolymer, or PEGT/PBT copolymer. In another alternative, polyalkylene glycol/polyester copolymers may be prepared as described in U.S. Pat. No. 3,908,201. It is to be understood, however, that the scope of the present invention is not to be limited to the specific copolymer hereinabove described, or to any particular means of synthesis.

Alternatives to the above-mentioned polyethylene glycol/polybutylene terephthalate copolymer may be prepared if one wishes to enhance the overall hydrophilic (or “soft”) or hydrophobic (or “hard”) properties of the polymer.

For example, if one wishes to enhance the hydrophobic properties or the polymer, a number of alternatives may be employed. Thus, in one embodiment, E is an ether, and preferably an ether having from 2 to 6 carbon atoms, more preferably from 2 to 3 carbon atoms. In another embodiment, the second component may include a mixture of ether moieties having 2 carbon atoms and 3 carbon atoms.

In one embodiment, diethylene glycol may replace butanediol in the mixture from which the polymer is synthesized. The extra oxygen in diethylene glycol renders the hydrophobic, or “hard”, component more hydrophilic, and may render the resulting polymer more flexible; i.e., less hard.

In other embodiments, alternatives to dimethylterephthalate (DMT) may be employed in the mixture from which the polymer is synthesized. In one embodiment, dimethyl -2,5-dihydroxy-terephthalate is employed instead of dimethylterephthalate. The presence of the two hydroxy groups renders the resulting “hard” component more hydrophilic. The greater hydrophilicity may favor hydrolysis in the “soft” component, as well as increase the probability of hydrolysis in the “hard” component. The two hydroxy groups provide increased water solubility, which results in a more rapid degradation. Also, the two hydroxy groups may provide possibilities for metabolic derivatization, which may result in lower toxicity. In addition, dimethyl -2,5- dihydroxy-terephthalic acid, which is liberated after degradation, may induce the calcification process.

In another embodiment, dimethylterephthalate -2,5-diglycinate ester or dimethoxyterephthalate -2,5- diglycinate ester may be employed in place of dimethylterephthalate. Such a diglycinate ester may result in a more hydrophilic structure for the “hard” component.

In yet another embodiment, amino dimethylterephthalate may be employed in the synthesis mixture. The use of amino dimethylterephthalate may provide increased hydrophilicity to the hard component. Also, the presence of the amino group may accelerate degradation as well as possibly inducing the calcification process.

In a further embodiment, the synthesis mixture may include diethylene glycol in place of butanediol, and one of the above-mentioned dimethylterephthalate derivatives in place of dimethylterephthalate.

In yet another embodiment, a polyethylene glycol “prepolymer” may be employed in the synthesis mixture instead of polyethylene glycol. Prepolymers of polyethylene glycol which may be employed include, but are not limited to, prepolymers of polyethylene glycol with glycine anhydride (2,5- piperazine dione), alloxan, uracil, 5,6- dihydrouracil, glycolic acid, and lactone groups having ester bonds, such as D-, L-isocitric acid lactone.

When D-, L-isocitric acid lactone is employed in the prepolymer, D-, L-isocitric acid is ultimately released upon degradation of the polymer. The released D-, L-isocitric acid may catalyze the hydrolysis of ester bonds, and may also enhance the calcification process by completing with calcium.

In yet another embodiment, the synthesis mixture may include diethylene glycol, a dimethylterephthalate derivative, and a polyethylene glycol prepolymer. In a preferred embodiment, the polymer is synthesized from a mixture of diethylene glycol, dimethoxyterephthalate -2,5- diglycinate ester, and a prepolymer of polyethylene glycol and D-, L-isocitric acid lactone ester. Such a polymer has the following structure:

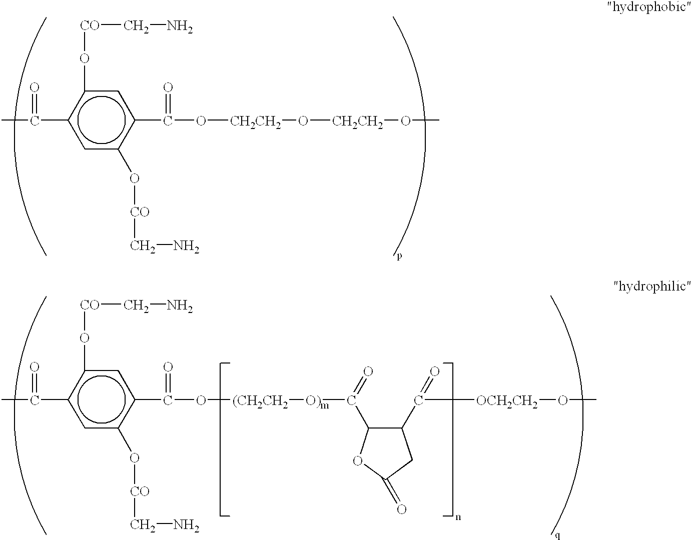

m is from about 10 to about 100; n is from 1 to about 10; p is from 1 to about 30; and q is from 1 to about 30.

In another embodiment, the polymer may include a polyphosphazene, to which the hydrophilic (“soft”) and hydrophobic (“hard”) components may be attached.

In general, polyphosphazenes have the following structural formula:

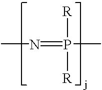

wherein R is an alkoxy, aryloxy, amino, alkyl, aryl, heterocyclic unit (e.g., imidazolyl), or an inorganic or organometallic unit.

In general, polyphosphazene derivatives may be synthesized from a precursor polymer known as poly (dichlorophosphazene) by macromolecular substitution of the chloride side moieties. The broad choice of side group structures which may be attached to the phosphorus atoms enables one to attach any of a variety of hydrophilic (“soft”) and hydrophobic (“hard”) components to the polyphosphazene. In addition, degradation inducers and other inert substituents may be attached to the polyphosphazene polymer backbone as well.

Thus, in accordance with another embodiment, the polymer has the following structural formula:

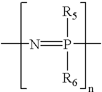

ₙ, wherein n is from about 50 to about 2,000, and each of R₅ and R₆ is selected from the group consisting of a first component, which, when contacted with calcium, calcium is deposited on or in the first component; a second hydrophobic component which imparts stability to the first component in water; a third component which induces degradation of the polymer; and a fourth inert component. At least about 10% of the total R₅ and R₆ moieties must be the first component.

Preferably, from about 10% to about 90% of the total R₅ and R₆ moieties are the first component, and from about 10% to about 70% of the total R₅ and R₆ moieties are the second component.

More preferably, from about 50% to about 70% of the total R₅ and R₆ moieties are the first component, and from about 30% to about 50% of the total R₅ and R₆ moieties are the second component.

In one embodiment, from about 10% to about 50% of the total R₅ and R₆ moieties may be the third component. In another embodiment, from about 10% to about 70% of the total R₅ and R₆ moieties may be the fourth component.

Hydrophilic, or “soft”, components which may be attached to the polyphosphazene polymer backbone include those hereinabove described, as well as methoxy polyethylene glycol, and amino-polyethylene glycol-monomethyl ether.

Hydrophobic, or “hard” components which may be attached to the polyphosphazene backbone include those hereinabove described, as well as phenylalanine ethyl ester, 2-amino-3-phenyl- -butyrolactone, and phenylalanine dimethyl glycolamide ester.

Substituents which induce degradation of the polymer, and which may be attached to the polyphosphazene polymer backbone include, but are not limited to, imidazole, 2-amino- -butyrolactone, and glycine dimethylglycolamide ester.

Other substituents which also may be attached to the polyphosphazene polymer backbone include inert substituents, such as, but not limited to, glycine ethyl ester, glycine dimethylamide ester, glycine methyl ester, amino-methoxy-ethoxy-ethane. The attachment of such inert compounds aids in enabling one to replace all available chlorines in the polydichlorophosphazene polymer backbone.

As representative examples of polymers which include polyphosphazenes to which are attached hydrophilic (“soft”) components, hydrophobic (“hard”) components, and possibly degradation inducers, and inert substituents, there may be mentioned the following (percentage values are indicative of the degree of substitution of the substituent in relation to the total degree of substitution):

1. 70% methoxy polyethylene glycol and 30% phenylalanine ethyl ester.

2. 70% amino-polyethylene glycol monomethyl ether and 30% phenylalanine dimethyl glycolamide ester.

3. 60% amino-polyethylene glycol monomethyl ether and 40% 2-amino [symbol] -butyrolactone.

4. 40% 2-amino-3-phenyl- [symbol] butyrolactone, 20% imidazole, and 40% amino-polyethylene glycol monomethyl ether.

5. 40% phenylalanine dimethyl glycolamide ester, 30% amino-polyethylene glycol monomethyl ether, and 30% glycine dimethylglycolamide ester.

6. 50% 2-amino-3-phenyl- [symbol] butyrolactone, 20% imidazole, 20% amino-polyethylene glycol monomethyl ether, and 10% glycine ethyl ester.

Applicants surprisingly have found that the polymers hereinabove described, such as, but not limited to, polyethylene glycol/polybutylene terephthalate copolymer (or PEGT/PBT copolymer), which bind to soft tissue and fibrous tissue, also bind to bone, which is a hard tissue. Such polymers are not only osteoconductive; i.e., the polymers provide for the proliferation of bone tissue upon the surface of the polymers; but bioactive as well; i.e., the polymers are bonded by bone tissue. Applicants have found that the polymers of the present invention form an electron-dense interface layer with bone which is continuous with the natural lamina limitans of bone. This constitutes evidence that the polymers of the present invention participate at least partially with normal bone metabolism where a lamina limitans (a cementing zone) occurs between two zones of bone deposited at different times or on top of bone where osteogenesis has ceased temporarily or definitively. Applicants have also found that in certain calcified sections of prosthetics formed from the polymers of the present invention, the lamina limitans interface between prosthetic and bone showed numerous crystals, which contained calcium and phosphorous and which resembled bone apatite crystals with respect to morphology and chemical composition.

Other bone-bonding substances, such as bioglasses, glass ceramics, and calcium phosphate ceramics (eg., hydroxyapatite), also showed an electron-dense interface layer with bone, thus suggesting that such an interface structure is associated with the bone-binding processes of these materials; however, such materials lack elastic properties. The presence of an electron-dense interface between bone and the materials of the present invention indicates that the material is chemically bonded by the bone by a process called bonding osteogenesis; i.e., the materials are bioactive.

The proportion of the amount of the soft component to the amount of the hard component in the polymer depends upon the desired characteristics of the prosthetic device. If one desires to form a prosthetic device which is elastomeric and which will calcify rapidly and thus bond to bone rapidly, one would form a device which has a greater amount of the soft component. If one desires to form a prosthetic device which has a more rigid structure, and can have a slower rate of calcification and less bone-bonding, one would form a device having a greater amount of the hard component.

The polymers of the present invention may include pores, although porosity is not a condition for bone-bonding. In one embodiment, the prosthetic device formed from the materials of the present invention has a surface which has a macroporosity of from about 30% to about 60% by volume. The term “macropores” as used herein means pores which have a diameter of from about 50μ to about 500μ. Preferably, the macropores have a diameter of from about 60μ to about 350μ, and more preferably from about 150μ to about 350μ. In one embodiment, macropores comprise over 90% of the total pore volume and micropores (less than 50μ in diameter) comprise under 10% of the total pore volume. The macropores, when present, enable the polymer to be ingrown by bone tissue. Thus, when the prosthetic device includes pores, bone-bonding is achieved both by bonding osteogenesis (establishment of a chemical bond) as well as by the growth of bone tissue into the pores of the polymer to provide a mechanical interlock. In one embodiment, pores can be obtained in situ by including salt particles in the shaped polymeric device. The salt particles are dissolved either before or after the device is implanted, thereby leaving pores in the device. The presence or absence of pores in the device, and the specific porosity of the device formed from the materials of the present invention is dependent upon the particular application of the device.

The devices of the present invention may also be pre-calcified prior to implantation, thereby providing for rapid bone bonding and bone ingrowth after implantation.

In addition, it is believed that an initial fixation of bone the polymers of the present invention may be achieved because of the swelling of the polymers, as a result of the water uptake by the polymers. Such swelling is particularly important when the polymers are used as coatings, whereby the coating becomes more flexible, thereby providing less stress shielding.

The polymers of the present invention may be formed into any of a variety of prosthetic devices. Examples of prosthetic devices which may be formed from the polymers of the present invention include, but are not limited to, prosthetic devices employed in head and neck surgery, such as, but not limited to, total and subtotal tympanic membrane replacements; total middle ear prostheses; coverings of middle ear bones, or middle ear mucosa to prevent adhesions; artificial ossicles; artificial palates; tympanic and sinus ventilation tubes; orthopedic implant coatings; distal portions of hip stems; mastoid repair devices; replacements for facia lata; ear canal walls; and closures of the nasal septum; devices used in plastic surgery and maxillofacial surgery, such as, but not limited to, bone augmentation with respect to the nose, chin, cheekbone, and eye socket; preformed noses; mandibles; skull augmentations; coatings of cochlear electrodes; tooth coatings; dental sheets; dental implant coatings; peridontal ligament replacement; osteotomy spacers; dental ridge augmentations; devices used in orthopedic surgery such as bone dressings, or bone-replacing or cartilage-replacing material; artificial joint coatings; fracture fixations; spinal fusion devices; artificial dowels; spinal fixations; disks; artificial ligaments; interstitial cartilage repair or replacement; anchor elements for ligament repair; swell fixations; and Hercules plugs; bone fillers; cartilage sheets; tubes to direct nerve growth; fracture bandages to hold bone pieces after compound fractures; skull fixations; burr hole plugs; cement plugs; and burr hole fillers.

The shape of the prosthetic devices may vary considerably, depending upon the particular application. Examples of shapes include, but are not limited to, films, woven and non-woven sheets, plates, screws, filaments for wrapping injured or fragmented bone, staples, “K” wire, and spinal cages.

When a prosthetic device of a copolymer material in accordance with the present invention is made, such device may be made in accordance with a variety of methods. In one embodiment, the device (such as an implant, for example) may be formed from sintered copolymer particles. When a film is employed, the copolymer may be liquefied in chloroform at a weight ratio of copolymer to chloroform of 1 to 10, and then fibers of the copolymer are spun. The fibers are then woven on a rotation axis to produce woven tubings which are cut lengthwise to produce films.

In another embodiment, a salt-casting technique may be employed. In this procedure, a copolymer is liquefied in chloroform at a weight ratio of copolymer to chloroform of 1 to 10. A certain amount of salt particles of desired sizes is then added to the copolymer solution. Salt particles having diameters of from 50μ to 500μ resulted in pores having diameters from 50μ to 500μ. The salt/copolymer solution is then either cast on a glass plate using a film-casting apparatus; fixed at the desired height (eg., about 200 microns) or used as a dip solution to obtain porous coatings. The ratio of salt to copolymer provides a desired porosity. For example, 6 g of salt (eg., sodium citrate or sodium chloride) per gram of copolymer results in films with porosities of about 50%.

If one desires to prepare a “dense” film; i.e., a film having pores no greater than 10μ in diameter, one may employ the casting technique hereinabove described except that salt particles are not added to the copolymer solution.

The prosthetic devices of the present invention may also be formed by injection molding or melt extrusion techniques. When one desires to prepare a porous material, one may admix salt particles, having sizes such as those hereinabove described, with the polymer prior to or upon feeding the polymer into the injection molding or melt extrusion device. If one desires to prepare a dense material, one does not add such particles to the polymer.

Alternatively, pores may be formed in the polymer by blending the polymer in the melt with a second polymer, such as, but not limited to, polyvinyl pyrrolidone, polyethylene glycol, or polycaprolactone, in order to form pores in the polymer. After blending, the second polymer forms a co-continuity with the first polymer. The second polymer then is washed out with a non-solvent for the first polymer. When preparing the dense layer, the salt particles, or the second polymer, are not included in the polymeric melt.

In another alternative, the polymer may be dissolved in chloroform, either with or without salt particles, depending on whether one wishes to prepare a porous device. The solution is the cast on a glass plate using a film-casting apparatus fixed at a desired height. Immediately after casting, the film is immersed in a non-solvent or a mixture of solvent and non-solvent. Depending upon actual conditions, pores can be formed, or pores may be preformed by the salt particles if they are employed.

In yet another alternative, the prosthetic devices of the present invention may be formed by gel casting techniques. In general, the polymer is dissolved in a solvent. The solution containing the polymer is then cast in a mold, and a gel is formed in situ. The shaped gel is removed from the mold, and the gel is then dried to obtain a solid material in thick sections. Examples of gel casting techniques are described in Coombes, et al., Biomaterials, Vol. 13, No. 4, pgs. 217-224 (1992) and in Coombes, et al., Biomaterials, Vol. 13, No. 5, pgs. 297-307 (1992).

In another alternative, porous materials may be formed through the use of foaming agents or blowing agents. A foaming agent or blowing agent is an agent that leads to the formation of pores in the polymer through the release of a gas at an appropriate time during processing. Examples of such foaming agents or blowing agents include, but are not limited to, nitrogen, carbon dioxide, chlorofluorocarbons, inorganic carbonate or bicarbonate salts, toluene sulfonyl hydrazide, oxybis (benzene sulfonyl hydrazide), toluene sulfonyl semicarbazide, and azodicarbonamide. In general, such agents are added prior to feeding the polymer to an injection molder or melt extrusion device. The amount of blowing agent added is dependent upon the pore size and the percent porosity desired in the formed prosthetic device.

In another alternative, a porous polymer material may be formed by forming initially a dense polymer, which is then subjected to laser treatment, whereby the laser penetrates the polymer and forms pores of a desired pore size.

In yet another alternative, a dense polymer may be mixed with a solvent, and the polymer is then melted under pressure. As the pressure is gradually removed, the polymer swells. During the swelling, pores are formed in the polymer.

In yet another alternative, a porous polymer may be made by an injection molding technique.

Depending upon the particular application of the prosthetic device, the device may be formed from a polymer which is entirely dense, or entirely porous, or which contains a combination of dense and porous components. When a combination of dense and porous components is employed, the dense and porous components may be formed in separate compartments of an injection molding or melt extrusion apparatus, and then coextruded and blended with or laminated to each other upon exiting the die of the appparatus. Laminates of dense and porous components may include 2 or more alternating dense and porous layers. Such alternating dense and porous layers may also be formed by salt casting, and then laminated after their formation.

Precalcified PEGT/PBT co-polymer feedstock (in the form of granules) can be injection molded to form precalcified injection molded products, or can be sintered to form precalcified sintered products.

It is also contemplated that the prosthetic devices of the present invention may be combined with additional materials such as, but not limited to, hydroxyapatite and polylactic acid, in which the materials of the present invention form a composite or a blend with such additional materials.

Also, in one alternative, the prosthetic devices of the present invention may be formed from more than one polymer of the present invention wherein the polymers have varying proportions of the soft and hard components.

The polymers of the present invention may also be used as dense or porous coatings for a prosthetic device such as those hereinabove described. The polymers may also be used as coatings for electrodes and subcutaneous devices, both of which are stabilized by bone adhesion.

It is also contemplated that the “soft” components hereinabove described may also be used as dense or porous coatings for a prosthetic device or as bone fillers. In one embodiment, the coating may be comprised of blocks of a polyalkylene glycol (such as polyethylene glycol) which are connected with a terephthalate. The terephthalate, however, does not become part of a segmented, or block copolymer.

In another embodiment, a non-elastomeric material such as, for example, a bioglass, a glass ceramic, or a calcium phosphate (hydroxyapatite) ceramic, insoluble salt particles, or metals, may be admixed with the polymer. Such filler materials may have a variety of shapes, such as, for example, spherical, or fibrous, or the materials may be irregular in shape. Preferably, the non-elastomeric material is a hydroxyapatite ceramic. The non-elastomeric material may be present in an amount of from about 5 vol. % to about 80 vol. %, based on the volume of the polymer, and preferably from about 20 vol. % to about 50 vol. %.

The present invention will now be described with respect to the following examples; however, the scope of the present invention is not intended to be limited thereby.

### EXAMPLE 1

A copolymer of polyethylene glycol terephthalate (PEGT) and polybutylene terephthalate (PBT), in which polyethylene glycol (PEG) has an average molecular weight (MW) of 1,000 and in which the copolymer has 80 wt. % of PEGT and 20 wt. % of PBT was made as follows: (In this example, and those that follow, DMT=dimethylterephthalate; 1,4-BD=1,4-butanediol; PEG=PEO-poly(ethylene glycol); Ti-cat.=tetra-butyltitanate, a catalyst):

DMT (313.8), 1,4-BD (209.7 g), PEG (709.2 g), and 1,3,5-trimethyl-2,4,6-tris(3,5-di-tert-butyl-4-hydroxybenzyl) benzene sold by Ciba-Geigy as Irganox 1330 antioxidant (5.00 g) are added to a 2 kg resin kettle equipped with a mechanical stirrer, a nitrogen inlet tube, a thermocouple, and a condenser. This system is continuously purged with nitrogen and is heated in 20 min. to 160° C. Upon reaching a temperature of 125° C., low speed stirring is started. When the reaction temperature is 160° C., the catalyst, tetra-butyltitanate (418.42 mg) is added in 10 ml 1,4-BD. The ester exchange reaction begins almost immediately, the stirring is intensified and the reaction temperature is increased over a 10 min period to 180° C. After about 1.5 hrs from the start the nitrogen purge is discontinued and a vacuum cycle is started. At this stage at least 80% of the theoretical amount of methanol is distilled. The pressure during the vacuum cycle is reduced in 20 min. to 220 mbar and is then further reduced to 60 mbar in 30 min. and maintained at this level for 10 min. by which time the theoretical amount of methanol has distilled. The pressure is then further rated reduced while the reaction temperature is increased over a 1 hr period to the 245° C. At a temperature of 180° C. and a pressure of 22 mbar, 1,4-BD is distilled. Polymerization is started. The vacuum cycle is maintained for 1.5 hrs. below 0.1 mbar. The polymer is then extruded and quenched in cold water followed by vacuum drying and grinding.

### EXAMPLES 2 -6

Copolymers of polyethylene glycol terephthalate/polybutylene terephthalate, in which PEG has an average MW of 2,000, and having

70 wt. % of PEGT and 30 wt. % of PBT (Example 2)

60 wt. % of PEGT and 40 wt. % of PBT (Example 3)

55 wt. % of PEGT and 45 wt. % of PBT (Example 4)

40 wt. % of PEGT and 60 wt. % of PBT (Example 5)

30 wt. % of PEGT and 70 wt. % of PBT (Example 6)

were all made according to Example 1 but with different quantities of DMT, 1,4-BD, PEG, and Ti-catalyst, which are given hereinbelow:

| | |
|---|---|
| 70/30: | DMT =385.1 g |
| | BD =259.6 g |
| | PEG =620.7 g |
| | Ti-cat. = 513.42 mgg |
| 60/40: | DMT = 456.4 g |
| | BD = 309.5 g |
| | PEG = 532.3 g |
| | Ti-cat. = 608.48 mg |
| 55/45: | DMT = 492.0 g |
| | BD - 334.4 g |
| | PEG = 487.9 g |
| | Ti-cat. = 492.02 mg |
| 40/60: | DMT = 599.1 g |
| | BD = 409.3 g |
| | PEG = 355.0 g |
| | Ti-cat. = 599.06 mg |
| 30/70: | DMT = 670.4 g |
| | BD = 459.3 g |
| | PEG = 266.3 g |
| | Ti-cat. = 670.47 mg |

The soft to hard ratio was assessed using proton nuclear magnetic resonance (NMR) and is shown in the following table. This table also includes the average molecular weight (Mw) of the copolymers from Examples 2-6, assessed by gel permeation chromatography (GPC).

| soft/hard | soft/hard | Mw |
|---|---|---|
| (PEG/PBT) | (NMR) | (GPC, in Daltons) |
| 70/30 (Ex.2) | 70.3/29.7 | 110,000 |
| 60/40 (Ex.3) | 60.3/39.7 | 96,000 |
| 55/45 (Ex.4) | 55.0/45.0 | 105,000 |
| 40/60 (Ex.5) | 42.6/57.4 | 111,000 |
| 30/70 (Ex.6) | 28.2/71.9 | 100,000 |

### EXAMPLE 7

A Series of PEGT/PBT copolymers were synthesized with a PEGT content of 70, 60, 55, 40, 30 wt. %. The copolymers were synthesized according to Examples 2 to 6. Both porous films (porosity 50%, pores 38-150 microns, 125 microns thick) and dense blocks (about 2×3×3 mm) were implanted in male Wistar rats (weight 200 g) subcutaneously and into the tibias. A total of 300 implants with survival times from 3 to 52 weeks were used. The implants were evaluated with light microscopy, image analysis, scanning-backscattered, and transmission electron microscopy, and X-ray microanalysis. For the demonstration of calcium in the copolymers, a combination of Sudan Black and alizarin red staining was used.

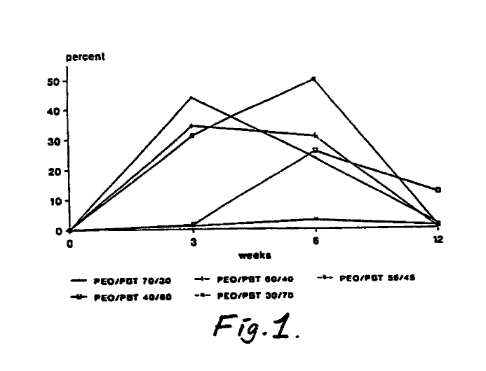

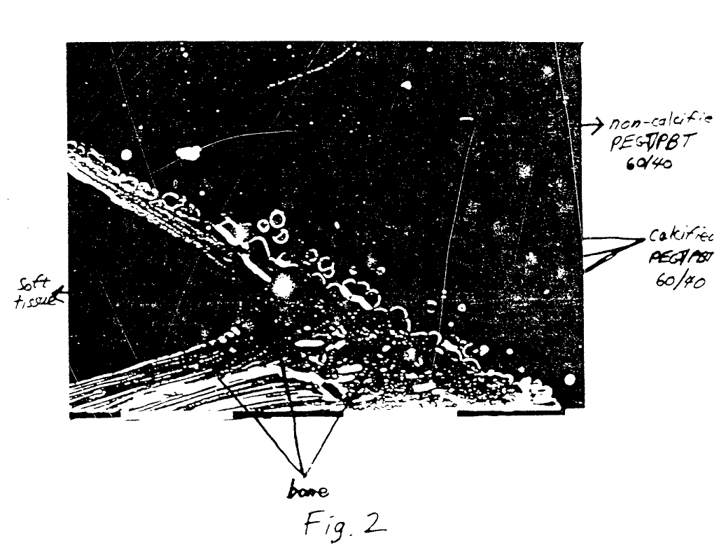

Sudan black/alizarin red staining an subcutaneous films showed that calcium was present in a large part of the polymers. This was confirmed by X-ray microanalysis. Using X-ray diffraction and electron diffraction, calcium phosphate deposition comprised of carbonated hydroxyapatite was demonstrated. Quantitative analysis of the stained polymer areas showed that most polymers revealed a similar calcification pattern in time. (FIG. 1). Initially no calcium was present, at a later stage a peak in calcification was reached (maximum Ca was 50%), and at the longest interval no noteworthy calcification areas were observed any more. The general pattern suggested that with the increase of PEO content the calcification peak occurred sooner and increased in height. With only 30% PEO minimal calcification was seen. Calcification of the polymers was also found near bone. Bone was deposited directly at the interface of all polymers. FIG. 2, which is a backscatter electron micrograph of the bone/copolymer interface, shows the continuity between the calcified copolymer PEGT/PBT 60/40 and the mineral phase of bone (hydroxyapatite). In the case of calcified copolymer, the copolymer/bone contact led to a continuity between the hydroxyapatite phase of the bone tissue and the calcium phosphate deposition on or within the copolymer. This continuity is responsible for the chemical bond across the bone/copolymer interface.

Using single spot x-ray microanalysis, the calcium to phosphate ratio (Ca/P ratio) was determined in: (i) the calcium phosphate depositions in the copolymers; (ii) the needle-shaped crystals in the lamina limitans-like interface between bone and copolymer; and (iii) the bone apatite. The Ca/P ratios in each instance were from about 1.6 to about 1.7. This suggests that the calcium phosphate depositions on or in the copolymer as well as the calcium phosphate depositions of the electron-dense interface were composed of hydroxyapatite, which is known to have a Ca/P ratio of 1.66-1.67 (atomic %).

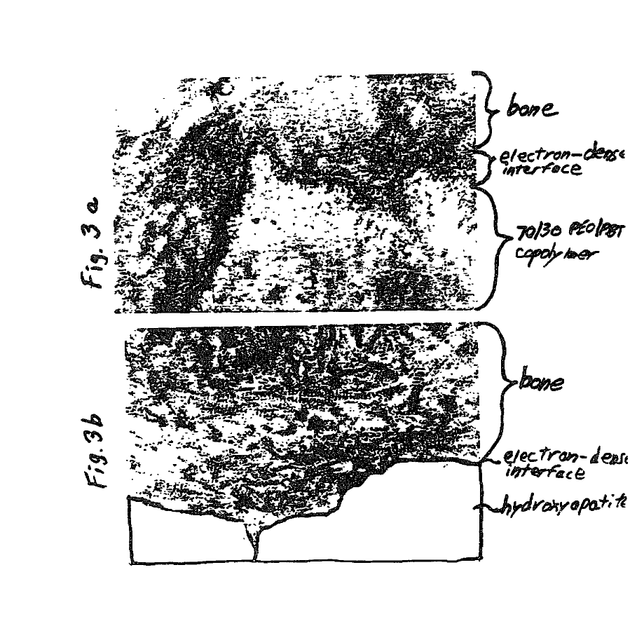

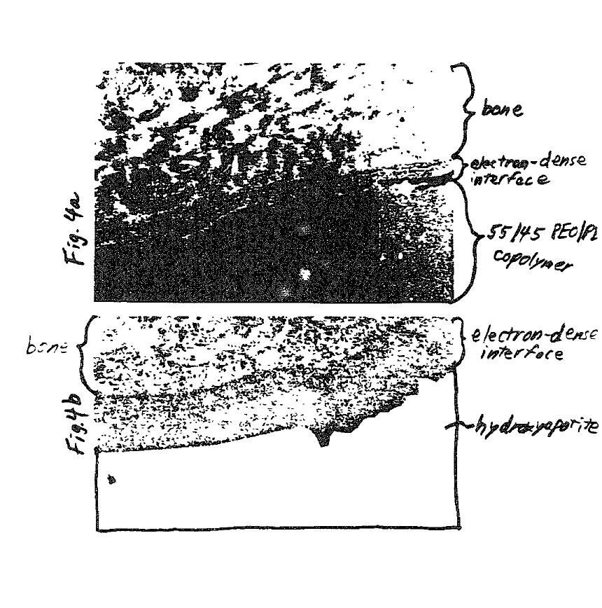

As will be described hereinbelow, decalcified material studied with the transmission electron microscope (See FIGS. 3a, 3b, 4a, and 4b) showed that the bone/copolymer interface was characterized by a granular electron-dense layer resembling the electron-dense (lamina limitans-like) interface between bone and hydroxyapatite as to morphology and composition. All materials with a bone contact showed an electron dense bonding zone very similar to that of hydroxyapatite.

As shown in the transmission electron micrographs of FIGS. 3a and 3b, an electron-dense interface was formed between the 70/30 PEO/PBT copolymer and bone which is similar to the electron-dense interface formed between bone and hydroxyapatite. This electron-dense interface was also found between bone and the 55/45 PEO/PBT copolymer, as shown in the transmission electron micrograph of FIG. 4a. Again, the electron-dense interface was similar to that found between bone and hydroxyapatite (FIG. 4b). Apparently depending on their PEO proportion, PEO/PBT copolymers calcify and behave in a way similar to hydroxyapatite as far as bone bonding is concerned. This suggests that calcium does not necessarily have to be present in an implant prior to implantation, but calcium adsorption or absorption after implantation might be sufficient for obtaining bonding osteogenesis.

### EXAMPLE 8

Two types of porous implants made of PEGT/PBT copolymers (70/30 and 55/45) were used in this study. The materials were synthesized according to Examples 2 and 4, respectively. Films (300 microns thick, pore size 38-150 microns, porosity 50%) , were cut into shapes of 5×15 mm² and folded into a triple layer of 5×5 mm². For comparative study, coralline hydroxyapatite ceramics (Interpore 200, Interpore International, Irvine, Calif., USA) were used. Rat bone marrow cells were prepared as described by Ohgushi et al. (J. Orthop. Res., Vol. 7, pg. 568 (1989)). Part of the implants were soaked in the marrow cell suspension. Implants with and without (negative control) bone marrow cells were implanted subcutaneously in the back of synergic Fisher rats. A total of 240 implants were used in 30 rats. The implants were harvested after 1, 2, 3, 4, 6, and 8 weeks after surgery. Undecalcified sections were studied by fluorochrome labeling (tetracycline, calcein). The sections were observed under light microscopy or fluorescence microscopy stained with Villanueva bone stain, Sudan Black, Alizarin Red and hematoxilin-eosin. The bone/implant interface was examined by SEM-EPMA (scanning electron microscopy combined with X-ray microanalysis) and transmission electron microscopy (TEM).

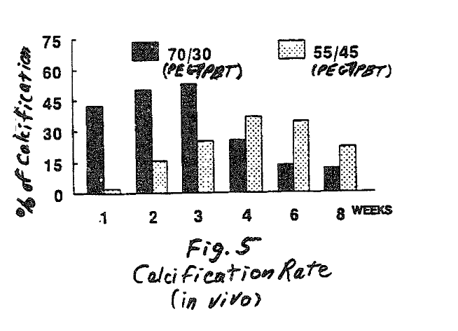

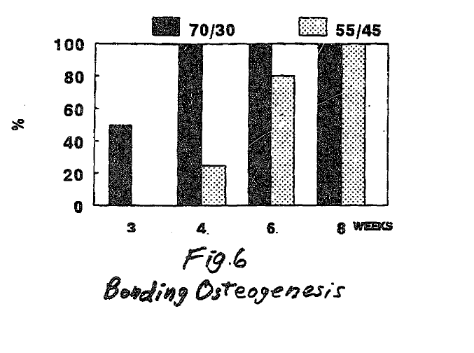

Both the 70/30 and the 55/45 implants showed areas of extensive calcification stained with Alizarin Red even one week after surgery. The calcification area was larger in the 70/30 polymer the first three weeks after implantation (see FIG. 5, calcification rate). All implants made of the copolymers under study showed calcification. However, only marrow cell loaded copolymer implants revealed new bone formation beginning three weeks postoperatively (see FIG. 6, bonding osteogenesis). Although the early bone formation started away from the implant surface, osteoblasts were deposited on the surface of calcified copolymer 70/30 and 55/45, and later, new bone was deposited. The bone formation proceeded from the surface of the copolymers in the direction of the center of the pores (according to the theory of bonding osteogenesis). Compared with 55/45, 70/30 copolymer showed the earliest appearance of calcification and bone deposition (FIGS. 5 and 6). Fluorochrome labeling confirmed that the bone formation started on the surface of the calcified implants made of 70/30 and 55/45 copolymers without an intervening layer of fibrous tissue, and that it proceeded to the center of the pores. SEM-EPMA analysis of both the bone/70/30 and the bone/55/45 interface showed high levels of calcium and phosphorus, in the (calcified) polymers, the bone, and the bone/polymer interface. This suggests a continuity (chemical bond) between the polymer-originated calcium phosphate deposition and the mineral matrix of living bone tissue. Undecalcified sections for TEM also showed bone bonding to the calcified 70/30 and 55/45 implants. The electron-dense interface described for bone/hydroxyapatite was also observed with these copolymer implants. Control hydroxyapatite (that is, without marrow cells), did not show any bone formation. SEM study of the hydroxyapatite surface showed (newly formed) calcium phosphate precipitates, two weeks after implantation. Hydroxyapatite implants combined with bone marrow cells (positive control) revealed primary bone formation on this newly-formed calcium phosphate layer. Fluorochrome labeling showed the consistent centripetal bone growth in all hydroxyapatite/marrow composites.

In this experiment, the PEO/PBT copolymers under study combined with marrow cells showed osteoblast deposition on the calcified polymer surface, and centripetal bone growth in a way similar to bioactive hydroxyapatite ceramics. 70/30 PEGT/PBT calcified first and showed the earliest bone deposition. These results suggest that PEGT/PBT copolymers 70/30 and 55/45 can sustain the bone marrow cell differentiation into osteogenic cells on its calcified surface and the differentiated cells (osteoblasts) cause bonding osteogenesis, apparently related to the calcification of these copolymers.

### EXAMPLE 9

Experiments were done with the following PEGT/PBT copolymers, which were prepared as disclosed in Examples 2-6: 70/30, 60/40, 55/45, 40/60, 30/70.

This study employs both a calvarial envelope technique which mimics the subperiosteal environment and a bone-marrow system, which allows information to be obtained on the differentiation and phenotypic expression of osteoblasts, related to the mineralization process. These two in vitro techniques are recognized to mimic the early aspects of the in vivo response to bioactive materials (J. E. Davies, CRC Handbook of Bioactive Materials, Yamamuro et al., ed. 1990, pg. 195). For the calvarial envelope method small polymer particles were used, smaller than 100 microns in diameter. Dense and porous films were inoculated with rat bone marrow cells. Cultures were maintained for 1, 2, 3, and 4 weeks. Light microscopical (LM) sections were stained with Alizarin Red and by the Von Kossa method. Further analysis was undertaken with SEM and TEM, Backscatter SEM and X-ray microanalysis (XRMA).

The results of these experiments were as follows:

Calvarial envelope system: Newly formed mineralized material deposited onto the partially calcified surface of 70/30, 60/40 and 55/45 samples was demonstrated in LM. In contrast a cellular layer was interposed with 40/60 and 30/70 particles and the advancing calcification front. SEM evaluation indicated a direct contact in a perpendicular fashion between calcified collagen fibers and a 55/45 particle. At an ultrastructural level a continuum between 70/30, 60/40 and 55/45 material and mineralized tissue was observed. Apatite-like crystals were seen penetrating the surface of the above specimens. These results were confirmed in backscatter SEM deposited bone-like tissue was observed in intimate contact with calcified areas in the 70/30, 60/40 and 55/45 surfaces. Analysis through the interfacial area with XRMA revealed a calcium and phosphorus signal.

Bone marrow system:

In SEM a calcified extracellular matrix was observed on 55/45 pressed plates. Linescans performed with XRMA revealed a continuous calcium and phosphorus signal through the interfacial area. Ultrastructural analysis indicated an intimate contact between mineralized deposition and the 60/40 and 55/45 samples, whereas in the bone marrow system, in contrast to the calvarial system, mineralized matrix was seen in contact with the 40/60 and 30/70 particles.

In both culture systems interfacial reactions similar to those observed in vivo seem reproducible for the range of materials. The evaluations indications indicate a continuum at an ultrastructural level between the 70/30, 60/40 and 55/45 surface and mineralized deposition. Distinct, however, was the composition of the 40/60 and 30/70 interface in the calvarial envelope system. Here, a cellular layer was present in close proximity to the polymer surface.

It is generally understood that the generation of a calcium and phosphorus rich outer surface of a biomaterial is a major requirement for bioactivity. In Bioglass™ such a layer is present shortly upon insertion, while in calcium phosphate ceramics this requirement is complied with through dissolution and reprecipitation of the bulk material. A possible explanation for the bioactivity of the polymers hereinabove described may lie in its hydrogelic properties which allow the polymer to swell and its soft segment to incorporate calcium ions. From the above findings it seems that the percentage of PEO may play a role in the surface calcification rate and the interfacial interaction. Apparently, a calcified surface is rapidly provided for the 70/30, 60/40 and 55/45 ratios, resulting in an intimate deposition of mineralized material onto the polymer. The polymers having the 40/60 and 30/70 ratios were also contacted with bone tissue; however, the deposition of the bone tissue was not continuous along the surface of the polymer.

### EXAMPLE 10

Dense implants were prepared from the 55/45 PEGT/PBT copolymer as synthesized according to Example 4, hydroxyapatite (HA) and tetracalcium phosphate (tetra-CP) as positive controls, and silicone rubber as a negative control.

72 dense blocks (2.5×2.5×2 mm³) equally distributed over the 4 materials under study were implanted with excessive clearance from the walls in cavities prepared through the lateral cortex of the tibia of mate Wistar rats (body weight 350 g). Animals were sacrificed after 3, 6, and 26 weeks and the tibias were fixed in 1.5% glutaraldehyde in buffer. Only specimens destined for light microscopy (LM) and transmission electron microscopy (TEM) were decalcified (4 weeks in a 10% EDTA solution in water containing the fixative). Part of the material from the 26 week survival period used for mechanical testing was processed for LM and TEM.

For push-out testing (3 weeks) the medial cortex was dissected from the tibia giving full view of the medial side of the implant. Using a Thermo Mechanical Analyser (Mettler TA 3000) at environmental temperatures, pull-out forces of up to the maximum of 2N were exerted on the medial side of the implants m (which were allowed to dry), while recording their movement. The force inducing a sudden shift of the implant indicating implant displacement was recorded as the push-out force during the pull-out tests (6 and 26 weeks) while using a Hounsfield 25 KN testing machine (pull-out rate of 1 mm/min), the implants were continuously soaked in saline. An adapted pair of tweezers was used to clamp the implant while pulling. The forces necessary to remove the implants from the tibiae or at which mechanical failure occurred were recorded.

Three weeks after the implantation the hydroxyapatite implants and the tetracalcium phosphate implants were bound to the bone in such a way that a “push-out”-pressure of about 1 mPa was not sufficient for removing the implants from the implantation bed. The silicone rubber implants were surrounded by an envelope of fibrous tissue and came loose during the preparation of the sample. The “bone-bonding” strength of the silicone rubber implant was less than about 0.01 MPa. With respect to the PEO/PBT-implants it is reported that said implants were bound to bone. The bone-bonding strength of the copolymer was in the range of 1 MPa. Six weeks and twenty-six weeks respectively after the implantation, the PEO/PBT-implants were bound to the bone with a bonding strength of about 4 MPa. In this respect it is noted that the limiting factor was not the bonding strength, but rather the strength of the polymer itself. All implants made of PEO/PBT fractured before they could be pushed out of the tibia. For the sake of completeness, it is reported that the implants made of hydroxyapatite and tetracalcium phosphate respectively tolerated a “push-out” pressure of about 7 MPa; at a higher pressure said implants also fractured.

Macroscopical and scanning electron-microscopical observations, within sections of bone viewed in polarized light, and ultrathin sections of bone studies by transmission electron microscopy showed bone with adherent polymeric fragments. Adhering fragments were seen for both normal and decalcified samples. Similar observations were made with implants made of both ceramics but not with those made of silicone rubber.

From this example it is clear that implants made of PEO/PBT are also chemically bound to bone, i.e., the contact zone of the copolymers with the bone was characterized by an electron-dense structure, the so-called “lamina limitans”-like interface.

The interface with bone was invariably characterized by an electron-dense layer continuous with the lamina limitans of bone. In decalcified sections, this layer was granular in appearance and up to 1000 nm thick. In undecalcified sections, the interface contained numerous crystals in contact with the polymer. They were shown by single spot microanalysis to contain calcium and phosphorus.

In this study it was shown that when bone came into contact with implants made of the copolymers, the resulting interface frequently consisted of an electron-dense granular layer. This laminar interface consisted of organic and inorganic components, the latter probably in the form of hydroxyapatite crystals. The interface was similar to that seen between bone and hydroxyapatite, both as to ultrastructural morphology and the presence of calcium and phosphorus. The bone/polymer interface was also morphologically similar to and frequently confluent with the natural lamina limitans of bone which occurs, for example, between two zones of bone deposited at different times. It is concluded that the electron-dense interface can be considered as the natural response of bone, constituting evidence that the polymers hereinabove described took part in normal bone metabolism resulting in the bond with bone.

### EXAMPLE 11

Copolymers of the following compositions:

70 wt. % polyethylene glycol terephthalate/30 wt. % polybutylene terephthalate;

60 wt. % polyethylene glycol terephthalate/40 wt. % polybutylene terephthalate;

55 wt. % polyethylene glycol terephthalate/45 wt. % polybutylene terephthalate;

40 wt. % polyethylene glycol terephthalate/60 wt. % polybutylene terephthalate; and

30 wt. % polyethylene glycol terephthalate/70 wt. % polybutylene terephthalate

were prepared as described in Examples 2 to 6. The polyethylene glycol had an average molecular weight of 1,000. Films of 100μ thickness were formed from the copolymers. Cultures of middle ear epithelium cells of a rat were grown on the copolymer films according to the procedure of Van Blitterswijk, et al., “Culture and Characterization of Rat Middle-ear Epithelium,” Acta Otolaryncol., Vol. 101, pgs. 453-466 (1986).

The epithelium cells cultured on these films for 7 and 12 days had the same morphology as cells cultured on tissue culture polystyrene. Best growth results of the epithelium cells were achieved with the 40/60 and 55/45 PEO/PBT films.

### EXAMPLE 12

Dense plates 2 mm thick were prepared from PEGT/PBT copolymer with a soft/hard ratio of 60/40, and an MW of PEG of 1000. The preparation of the particular 60/40 copolymer is disclosed in Example 3.

The plates (thickness 2 mm) were attached to the bottom of a culture dish. The culture dishes were sterilized by ultraviolet radiation and soaked in four different sterile solutions for 1, 2, 4 and 8 days. The medium employed was α-Minimal Essential Medium, containing 1.36 mM CaCl₂ and 1.00 mM NaH₂PO₄; 0.68M CaCl₂ and 0.29M NaH₂PO₄; 1.00M Ca(NO₃)₂ and Aqua dest. After the soaking procedure the plates were rinsed with Aqua dest for 10 minutes and dried. Bone marrow cells of the femora of 100-120 gram male Wistar rats were isolated and cultured according to Maniatopoulos et al., Cell Tiss. Res., Vol 254, pg. 317 (1988). Cells of the second passage were seeded on the polymer plates and cultured for 8, 10, 15 and 22 days. As a control some plates were “cultured” without cells to see the effect of the culture medium on the polymer plates. Plates soaked in the saturated Ca/P solution but not cultured were examined to determine the effect of the culture procedure.

The plates with the cells were rinsed in PBS and fixed in 1.5% glutaradehyde in 0.14M sodium cacodylate (pH 7.4) for 1 hour at 4° C.

The plates were postfixed with 1% OsO₄ and 1.5% K₄Fe(CN)₆ for 1 hour at 4° C., rinsed in PBS and dehydrated through a graded series of ethanol and embedded in an epoxy resin. The specimens were examined with light microscopy (LM) (Alizarin Red staining), transmission electron microscopy (TEM), analytical electron microscopy (AEM), and X-ray microanalysis (XRMA).

Semi- and ultrathin sections were made on an LKB ultramicrotome. Semithin sections for LM were stained with Alizarin-red for calcium. Ultrathin sections were stained with uranyl acetate and lead citrate and examined at 80 kV in a Philips EM 201. Sections used for AEM were not stained. For XRMA, epoxy blocks were coated with carbon and examined with a Tracor Northern X-ray microanalysing system attached to a Philips S 525 SEM.

The results were as follows:

LM: After 22 days of culture, Alizarin-red stained sections of the plates soaked in α-MEM, Ca(NO₃)₂ and Aqua dest solutions showed no positive staining for calcium in the PEGT/PBT plates or at the interface with the cells. However, the PEGT/PBT plates soaked in CaCl₂ and NaH₂PO₄ solutions showed extensive positive staining for calcium in the material. Control plates soaked in the Ca/P solution for 8 days, but cultured without cells, also showed a positive staining for calcium.

TEM: Ultrathin sections of plates soaked in CaCl₂ and Na₂PO₄ solution showed the presence of small crystals in the material, but not at the interface. These crystals were present at a depth of 10 μm and more. Large crystallization spots were observed. Analysis of the crystals by AEM showed the presence of calcium and phosphorus.

XRMA: Calcium and phosphorus were detected with XRMA spot analysis in the material. Linescans and X-ray mappings showed that calcium and phosphorus were present in plates which have been soaked in Ca/P solution, but could not be detected in plates soaked in Ca(NO₃)₂ and Aqua dest. In plates soaked in -MEM, calcium and phosphorus are present at the interface, but not in the bulk material. This can imply the presence of a Ca/P rich surface layer.

Soaking PEGT/PBT 55/45 copolymer discs in a supersaturated calcium chloride and sodium hydrogen phosphate solution result in the formation of calcium and phosphorus containing crystals in the polymer, approximately 10 microns below the surface of the discs as seen in the culture experiments. These crystals were also found in the control discs, which were cultured without the marrow cells. This indicates that the formation of these crystals is certainly not a fully cellular process. The PEGT/PBT copolymer under study probably incorporates calcium ions and phosphate ions from the supersaturated calcium phosphate solution, which enables the formation of calcium phosphate crystals under culture conditions.

In a second experiment, dense plates 2 mm thick made of PEGT/PBT copolymers with 80, 70, 60, 55, 40, and 30 wt. % of PEG having a molecular weight of 1000 (which were prepared according to Examples 1-6) were first soaked in a calcium chloride solution (4 M in distilled water, 2 days at room temperature) and then for 2 days at room temperature in an 8M disodium hydrogen phosphate solution in distilled water. After being immersed in either solution, samples were thoroughly rinsed with distilled water.

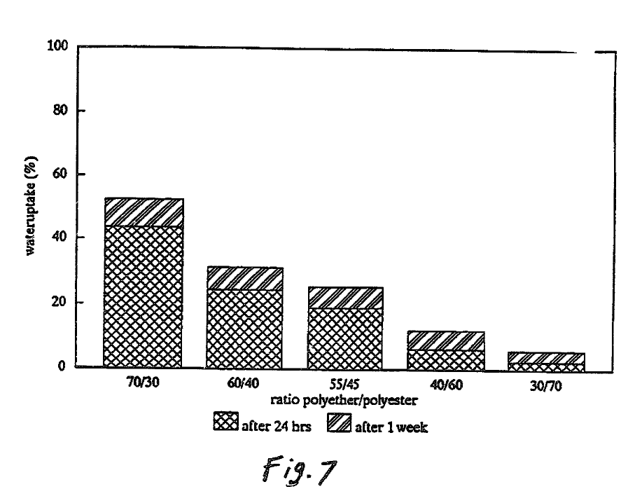

The samples were tested for water uptake according to ASTM Designation DS70-81, “Standard Test Method for Water Absorption of Plastics” (December 1981, reapproved 1988). Water uptake for the samples is shown in FIG. 7.

All samples were also found to contain calcium phosphate crystals. The amount of calcium phosphate crystals contained in the samples is directly related to water uptake by the polymer. Calcium phosphate deposition was the most extensive with the 80/20 material (not shown in FIG. 7) and the 70/30 material. Calcification was seen both in the polymers as well as on the dense polymer plates. Calcification, although present, was the least extensive with the 40/60 and 30/70 materials. It was restricted predominantly to the surface of the plates. Using X-ray diffraction techniques, the precipitated salt was shown to be composed predominantly of monotite (calcium hydrogen phosphate or CaHPO₄), although other calcium phosphate salts were also seen. Similar calcification experiments were done with sodium dihydrogen phosphate with comparable results. Brushite (CaHPO₄.2H₂O) was now the predominant calcium salt, although other calcium salts, such as hydroxyapatite and tetracalcium phosphate, were present as well.

### EXAMPLES 13-18

Copolymers of PEGT/PBT, including PEG of different molecular weights, and PBT, having 55 wt. % of PEGT and 45 wt. % of PBT, were made according to Example 1 but with different quantities of DMT, BD, PEG, and Ti-catalyst:

| | | |
|---|---|---|
| Ex. 13 | PEG 300: | DMT = 646.5 g |
| | | BD = 442.6 g |
| | | PEG = 384.9 g |
| | | Ti-cat. = 646.51 mg |
| Ex. 14 | PEG 600: | DMT = 544.0 g |
| | | BD = 370.8 g |
| | | PEG = 453.3 g |
| | | Ti-cat. = 544.00 mg |
| Ex. 15 | PEG 1500: | DMT = 462.9 g |
| | | ED = 314.1 9 |
| | | PEG = 507.3 g |
| | | Ti-cat. = 462.93 mg |
| Ex. 16 | PEG 2000: | DMT = 447.5 g |
| | | BD = 303.3 g |
| | | PEG = 517.6 g |
| | | Ti-cat. = 447.5 mg |
| Ex. 17 | PEG 3000: | DMT = 431.3 g |
| | | BD = 292.0 g |
| | | PEG = 528.3 g |
| | | Ti-cat. = 431.43 mg |
| Ex. 18 | PEG 4000: | DMT = 423.1 g |
| | | BD = 286.2 g |
| | | PEG = 533.9 g |
| | | Ti-cat. = 423.14 mg |

### EXAMPLE 19

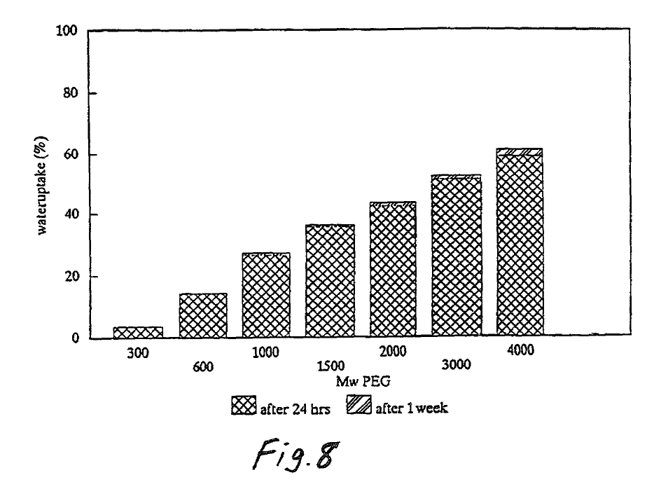

The copolymers of Examples 13-18 were tested for water uptake according to ASTM Designation D570-81 as hereinabove described in Example 12. Water uptake of the polymers is shown in FIG. 8. As shown in FIG. 8, the PEGT/PBT copolymers having 55 wt. % of PEGT, and of which the molecular weight of the PEGT was 600 or more, took up more than about 10% by weight of water.

The copolymers of Examples 13-18 were also studied for in vitro calcification using the method described in Example 12, second method. The samples which showed calcification were those which had a water uptake of at least about 10%; i.e., those samples in which the molecular weight of the PEG was 600 or more, such results suggest a positive correlation between hydrophilicity (or water uptake, or hydrogel behavior) and calcification.

### EXAMPLE 20

PEGT/PBT 55/45 copolymers having a molecular weight of PEG of 1,000 were synthesized as described in Example 4, and PEGT/PBT 55/45 copolymers having a molecular weight of PEG of 1,500 were synthesized as described in Example 15. 55/45 PEGT/PBT copolymers were synthesized as described in Example 4. The copolymers were then cryogenically grinded (in liquid nitrogen) to form particles less than 1 mm in size, and sieved to obtain particles having sizes from about 300μ to about 500μ. The particles are placed in a mold, which is heated to melt the superficial parts of the particles. After cooling, the particles had partially fused, resulting the formation of implants 2 mm in diameter and several cm long. The implants have a porosity of about 50% and pore sizes of from about 100μ to about 500μ. The implants were cut into pieces about 3 mm long, and implanted either by press-fitting into cavities prepared through the lateral cortex of the tibias of four male Wistar rats according to the procedure of Example 10 (for the PEG-1,000 copolymer), or subcutaneously (for the PEG-1,500 copolymer). The rats were sacrificed 4 weeks after implantation and the tibias and subcutaneous implants were processed for light microscopy as described in Example 10 and Example 7, respectively. Light microscopy of the tibial implants showed that after 4 weeks about 50% of the pore volume was occupied by bone tissue and about 50% of the pore volume was occupied by fibrous tissue. Bone tissue was frequently in contact with the 55/45 PEGT/PBT copolymer.

Light microscopy of the subcutaneous implants showed that the pores of the copolymers were filled with fibrous tissue. The copolymers also showed calcification.

It is to be understood, however, that the scope of the present invention is not to be limited to the specific embodiments described above. The invention may be practiced other than as a particularly described and still be within the scope of the accompanying claims.

What is claimed is:

1. A prosthetic device capable of binding to bone, comprising:
a polymer, said polymer being a polymer which, when contacted with a calcium salt, calcium is deposited on or in said polymer, said polymer including a first component, which when contacted with calcium, calcium is deposited on or in said first component; and a second hydrophobic component which imparts stability to the first component in water.

2. The device of claim 1 wherein said first component is capable of absorbing water.

3. The device of claim 2 wherein said first component is in the form of a hydrogel.

4. The device of claim 3 wherein said first component includes a component selected from the group consisting of polyethers; polyamines; polyvinyl acetate; polyvinyl alcohol; polyvinyl pyrrolidone; polyacrylic acid; poly (hydroxyethyl methacrylate); thioethers; and a polypentapeptide selected from the group consisting of: (Val Pro Gly Val Gly)ₙVal; (Oly Val Gly Val Pro)ₙ; and (Gly Val Gly Val Pro)ₙVal, wherein n is at least 2.

5. The device of claim 4 wherein said first component includes a polyether.

6. The device of claim 5 wherein said polyether is a polyalkylene glycol.

7. The device of claim 6 wherein said polyalkylene glycol is polyethylene glycol.

8. The device of claim 1 wherein said second component is selected from the group consisting of urethanes, amides, and esters.

9. The device of claim 8 wherein said second component is an ester.

10. The device of claim 9 wherein said ester has the following structural formula: 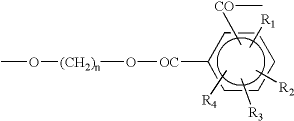
, wherein n is from 2 to 8, and each of R₁, R₂, R₃, and R₄ is hydrogen, chlorine, nitro-, or alkoxy, and each of R₁, R₂, R₃, and R₄ is the same or different.

11. The device of claim 10 wherein each of R₁, R₂, R₃, and R₄ is hydrogen.

12. A prosthetic device comprising a polymer, a polymer being a segmented thermoplastic polymer comprising a plurality of recurring units of a first component and of a second component, wherein said first component comprises from about 20 wt. % to about 98 wt. %, based upon the weight of said polymer, of units having the formula:
—OLO—CO—R—CO—, wherein L is selected from the group consisting of a divalent radical remaining after removal of terminal hydroxyl groups from a poly (oxyalkylene) glycol; and a polymer including a first moiety and a second moiety, said first moiety being a polyalkylene glycol and said second moiety being selected from the group consisting of glycine anhydride, alloxan, uracil, 5,6-dihydrouracil, glycolic acid, lactic acid, and lactones, and said second component comprises from about 2 wt. % to about 80 wt. %, based upon the weight of said polymer, of units of the formula:
—OEO—CO—R—CO—, wherein E is an organic radical selected from the group consisting of a substituted or unsubstituted alkylene radical having from 2 to 8 carbon atoms, and a substituted or unsubstituted ether moiety; and R is a substituted or unsubstituted divalent radical remaining after removal of carboxyl groups from a dicarboxylic acid.

13. The device of claim 12 wherein L is a divalent radical remaining after removal of terminal hydroxyl groups from a poly (oxyalkylene) glycol.

14. The device of claim 13 wherein said poly (oxyalkylene)glycol is selected from the group consisting of poly (oxyethylene) glycol, poly (oxypropylene) glycol, and poly (oxybutylene) glycol.

15. The device of claim 14 wherein said poly (oxyalkylene) glycol is poly (oxyethylene) glycol.

16. The device of claim 12 wherein E is an alkylene radical having from 2 to 8 carbon atoms.

17. The device of claim 16 wherein E is an alkylene radical having from 2 to 4 carbon atoms.

18. The device of claim 17 wherein said second component is selected from the group consisting of polyethylene terephthalate, polypropylene terephthalate, and polybutylene terephthalate.

19. The device of claim 18 wherein said second component is polybutylene terephthalate.

20. The device of claim 12 wherein L is a polymer including a first moiety and a second moiety, said first moiety being a polyalkylene glycol and said second moiety being selected from the group consisting of glycine anhydride, alloxan, uracil, 5,6-dihydrouracil, glycolic acid, lactic acid, and lactones.

21. The device of claim 20 wherein said first moiety is polyethylene glycol and said second moiety is a lactone.

22. The-device of claim 21 wherein said lactone is D,L-isocitric acid lactone.

23. The device of claim 12 wherein E is an ether.

24. The device of claim 23 wherein said ether has from 2 to 6 carbon atoms.

25. A prosthetic device capable of binding to bone, comprising:
a polymer including
a first component comprising a polyalkylene glycol; and
a second hydrophobic component which imparts stability to the first component in water.

26. The device of claim 25 wherein said polyalkylene glycol is selected from the group consisting of polyethylene glycol, polypropylene glycol, and polybutylene glycol.

27. The device of claim 26 wherein said polyalkylene glycol is polyethylene glycol.

28. The device of claim 25 wherein said second component is a polyester.

29. The device of claim 28 wherein said polyester is selected from the group consisting of polyethylene terephthalate, polypropylene terephthalate, and polybutylene terephthalate.

30. The device of claim 29 wherein said polyester is polybutylene terephthalate.

31. A process for providing an animal with a prosthetic, comprising:
implanting into an animal adjacent to bone of the animal a prosthetic comprising a polymer which, when contacted with a calcium salt, calcium is deposited on or in said polymer, said polymer including a first component which, when contacted with calcium, calcium is deposited on or in said first component, and a second hydrophobic component which imparts stability to the first component in water.

32. The process of claim 31 wherein said first component is capable of absorbing water.

33. The process of claim 32 wherein said first component is in the form of a hydrogel.

34. The process of claim 33 wherein said first component includes a component selected from the group consisting of polyethers; polyamines; polyvinyl acetate; polyvinyl alcohol; polyvinyl pyrrolidone; polyacrylic acid; poly (hydroxyethyl methacrylate); thioethers; and a polypentapeptide selected from the group consisting of (Val Pro Gly Val Gly)ₙVal; (Gly Val Gly Val Pro)ₙ; and (Gly Val Gly Val Pro)ₙVal, wherein n is at least 2.

35. The process of claim 34 wherein said first component includes a polyether.

36. The process of claim 35 wherein said polyether is a polyalkylene glycol.

37. The process of claim 31 wherein said second component is selected form the group consisting of polyurethanes, polyamides, and polyesters.

38. The process of claim 37 wherein said second component is a polyester.

39. The process of claim 38 wherein said polyester is formed from ester units having the following structural formula: 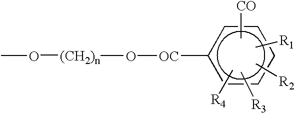
, wherein n is from 2 to 8, and each of R₁, R₂, R₃, and R₄ is hydrogen, chlorine, nitro-, or alkoxy, and each of R₁, R₂, R₃, and R₄ is the same or different.

40. The process of claim 39 wherein each of R₁, R₂, R₃, and R₄ is hydrogen.

41. A process for providing an animal with a prosthetic, comprising:
implanting into an animal adjacent to bone of the animal a prosthetic comprising a polymer, wherein said polymer is a segmented thermoplastic polymer comprising a plurality of recurring units of said first component and of said second component, wherein said first component comprises from about 20 wt. % to about 98 wt. %, based upon the weight of said polymer, of units having the formula:
—OLO—CO—R—CO—, wherein L is selected from the group consisting of a divalent radical remaining after removal of terminal hydroxyl groups from a poly (oxyalkylene) glycol; and a polymer including a first moiety and a second moiety, said first moiety being a polyalkylene glycol and said second moiety being selected from the group consisting of glycine anhydride, alloxan, uracil, 5,6-dihydrouracil, glycolic acid, lactic acid, and lactones, and said second component comprises from about 2 wt. % to about 80 wt. %, based upon the weight of said polymer, of units of the formula:
—OEO—CO—R—CO—, wherein E is an organic radical selected from the group consisting of a substituted or unsubstituted alkylene radical having from 2 to 8 carbon atoms, and a substituted or unsubstituted ether moiety; and R is a substituted or unsubstituted divalent radical remaining after removal of carboxyl groups from a dicarboxylic acid.

42. The process of claim 41 wherein L is a divalent radical remaining after removal of terminal hydroxyl groups from a poly (oxyalkylene) glycol.

43. The process of claim 42 wherein said poly (oxyalkylene) glycol is selected from the group consisting of poly (oxyethylene) glycol, poly (oxypropylene) glycol, and poly (oxybutylene) glycol.

44. The process of claim 43 wherein said poly (oxyalkylene) glycol is poly (oxyethylene) glycol.

45. The process of claim 41 wherein E is an alkylene radical having from 2 to 8 carbon atoms.

46. The process of claim 45 wherein E is an alkylene radical having from 2 to 4 carbon atoms.

47. The process of claim 46 wherein said second component is selected from the group consisting of polyethylene terephthalate, polypropylene terephthalate, and polybutylene terephthalate.

48. The process of claim 47 wherein said second component is polybutylene terephthalate.

49. The process of claim 41 wherein L is a polymer including a first moiety and a second moiety, said first moiety being a polyalkylene glycol and said second moiety being selected from the group consisting of glycine anhydride, alloxan, uracil, 5,6-dihydrouracil, glycolic acid, lactic acid, and lactones.

50. The process of claim 49 wherein said first moiety is polyethylene glycol and said second moiety is a lactone.

51. The process of claim 50 wherein said lactone is D,L-isocitric acid lactone.

52. The process of claim 41 wherein E is an ether.

53. The process of claim 52 wherein said ether has from 2 to 6 carbon atoms.

54. A process for providing an animal with a prosthetic, comprising:
implanting into an animal adjacent to bone of the animal a prosthetic comprising a polymer including a first component comprising a polyalkylene glycol; and
a second hydrophobic component which imparts stability to the first component in water.

55. The process of claim 54 wherein said polyalkylene glycol is selected from the group consisting of polyethylene glycol, polypropylene glycol, and polybutylene glycol.

56. The process of claim 55 wherein said polyalkylene glycol is polyethylene glycol.

57. The process of claim 54 wherein said second component is a polyester.

58. The process of claim 57 wherein said polyester is selected from the group consisting of polyethylene terephthalate, polypropylene terephthalate, and polybutylene terephthalate.

59. The process of claim 58 wherein said polyester is polybutylene terephthalate.

60. The device of claim 1 wherein said polymer has the following structural formula: 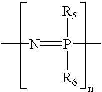
, wherein n is from about 50 to about 2,000, and each of R₅ and R₆ is selected from the group consisting of a first component, which when contacted with calcium, calcium is deposited on or in said first component; a second hydrophobic component which imparts stability to the first component in water; a third component which induces degradation of said polymer; and a fourth inert component, with the proviso that at least about 10% of the total R₅ and R₆ moieties are said first component.

61. The device of claim 60 wherein from about 10% to about 90% of the total R₅ and R₆ moieties are the first component, and from about 10% to about 70%. of the total R₅ and R₆ moieties are the second component.

62. The device of claim 61 wherein from about 50% to about 70% of the total R₅ and R₆ moieties are the first component, and from about 30% to about 50% of the total R₅ and R₆ moieties are the second component.

63. The device of claim 60 wherein from about 10% to about 50%, of the total R₅ and R₆ moieties are said third component.

64. The device of claim 60 wherein from about 10% to about 70% of the total R₅ and R₆ moieties are said fourth component.

65. The process of claim 31 wherein said polymer has the following structural formula: 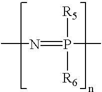
, wherein n is from about 50 to about 2,000, and each of R₅ and R₆ is selected from the group consisting of a first component, which when contacted with calcium, calcium is deposited on or in said first component; a second hydrophobic component which imparts stability to the first component in water; a third component which induces degradation of said polymer; and a fourth inert component, with the proviso that at least about 10% of the total R₅ and R₆ moieties are said first component.

66. The process of claim 65 wherein from about 10% to about 90% of the total R₅ and R₆ moieties are the first component, and from about 10% to about 70% of the total R₅ and R₆ moieties are the second component.

67. The process of claim 66 wherein from about 50% to about 70% of the total R₅ and R₆ moieties are the first component, and from about 30% to about 50% of the total R₅ and R₆ moieties are the second component.
